(19) United States
(12) Patent Application Publication
(10) Pub. No.: US 2011/0152917 A1
(43) Pub. Date: Jun. 23, 2011

(54) INTRAVASCULAR FILTER RESTRAINING DEVICE

(75) Inventor: Jim Anderson, Minneapolis, MN (US)

(73) Assignee: Boston Scientific Scimed, Inc., Maple Grove, MN (US)

(21) Appl. No.: 12/644,338

(22) Filed: Dec. 22, 2009

(51) Int. Cl. A61M 29/00

(52) U.S. Cl. 606/200

(57) ABSTRACT

A filtering device includes a filter that captures embolic material, a plurality of biased support struts that expand outwardly to deploy the filter, and a restraining mechanism. The restraining mechanism applies a restraining force that prevents the plurality of biased support struts from expanding outwardly to deploy the filter and includes a sleeve with one or more sleeve support bands biased to expand from a first position to a second position. An actuating member cooperates with a portion of the restraining mechanism to allow the sleeve support band to expand and release the restraining force.

### FIELD OF THE INVENTION

This disclosure relates generally to implantable medical devices and more particularly relates to intravascular filter devices that are configured for percutaneous insertion into the blood vessel of a patient.

### DESCRIPTION OF RELATE ART

Human blood vessels may become occluded or blocked by plaque, thrombi, or other material that reduce the blood carrying capacity of the vessel. Should the blockage occur at a critical place in the circulatory system, serious injury or death may result. Medical intervention can be performed when such an occlusion is detected.

Several procedures are now used to open these occluded vessels, including angioplasty, atherectomy, and stenting. Angioplasty is a well known procedure that uses an inflatable balloon to dilate the occlusion. Atherectomy involves removing the matter occluding the vessel through one of a variety of means, and the process may sometimes be repeated until a sufficient amount of material has been removed to restore blood flow to an acceptable level. Stenting involves temporarily or permanently implanting a substantially cylindrical tube or mesh sleeve into the occluded area of a vessel to radially open the lumen of the vessel. During any of these procedures, material may be separated from the wall of the blood vessel. This separated material can enter the bloodstream, and may be large enough to occlude smaller downstream blood vessels, potentially blocking blood flow to tissue. Intravascular filters are commonly used to capture this separated material during the procedure.

However, it is possible to dislodge some of this material during the placement or introduction of the devices used in the procedure. Therefore, there exists a need to improve the profile and operation of the devices during the insertion process.

### BRIEF SUMMARY OF THE INVENTION

The disclosure pertains generally to intravascular filter devices such as embolic protection filters and pertains more particularly to intravascular filters with improved operational characteristics.

Accordingly, an illustrative but non-limiting example may be found in a restraining mechanism for an intravascular filtering device that may include a sleeve and a sleeve support band fixedly attached thereto, the sleeve adapted to wrap around the filter and apply a restraining force to a plurality of support struts of the filtering device when the sleeve support band is in a first position. The device may also include an actuating member slidably engaged with at least two tubular members attached to the sleeve. The actuating member may be configured to release the restraining force and allow expansion of the sleeve support band toward a second position upon moving the actuating member in a proximal direction. The sleeve may be non-removably coupled to the actuating member. The sleeve support band may be non-removably coupled to at least one of the plurality of support struts. The filtering device may also include a second sleeve support band having any or all of the same characteristics as the first. The sleeve and at least two tubular members may have any one or more of a number of additional characteristics including material type, coating, and integration with other components.

Another illustrative but non-limiting example of may be found in a restraining mechanism for an intravascular filtering device that may include a sleeve and a sleeve support band fixedly attached thereto, the sleeve adapted to wrap around the filter and apply a restraining force to a plurality of support struts of the filtering device when the sleeve support band is in a first position. The device may also include an actuating member slidably engaged with at least two tubular members attached to the support band. The actuating member may be configured to release the restraining force and allow expansion of the sleeve support band toward a second position upon moving the actuating member in a proximal direction. The sleeve may be non-removably coupled to the actuating member. The sleeve support band may be non-removably coupled to at least one of the plurality of support struts. The filtering device may also include a second sleeve support band having any or all of the same characteristics as the first. The sleeve and at least two tubular members may have any one or more of a number of additional characteristics including material type, coating, and integration with other components.

The above summary is not intended to describe each disclosed embodiment or every implementation of the claimed invention. The Figures, Detailed Description, and Examples which follow more particularly exemplify these embodiments.

### BRIEF DESCRIPTION OF THE DRAWINGS

FIG. 1—illustrates a perspective view of an exemplary restraining mechanism according to one embodiment;

FIG. 2A—illustrates a cross-sectional view of the restraining mechanism of FIG. 1 in a first position according to one exemplary embodiment;

FIG. 2B—illustrates a cross-sectional view of the restraining mechanism of FIG. 1 in a second position according to one exemplary embodiment;

FIG. 3—illustrates a perspective view of an exemplary restraining mechanism according to one embodiment;

FIG. 4A—illustrates a detailed perspective view of a portion of an exemplary restraining mechanism according to one embodiment;

FIG. 4B—illustrates a detailed perspective view of a portion of an exemplary restraining mechanism according to one embodiment;

FIG. 5—illustrates a perspective view of an exemplary restraining mechanism according to one embodiment;

FIG. 6A—illustrates a cross-sectional view of the restraining mechanism of FIG. 5 in a first position according to one exemplary embodiment;

FIG. 6B—illustrates a cross-sectional view of the restraining mechanism of FIG. 5 in a second position according to one exemplary embodiment;

FIG. 7A—illustrates a detailed perspective view of a portion of an exemplary restraining mechanism according to one embodiment; and

FIG. 7B—illustrates a detailed perspective view of a portion of an exemplary restraining mechanism according to one embodiment.

### DETAILED DESCRIPTION

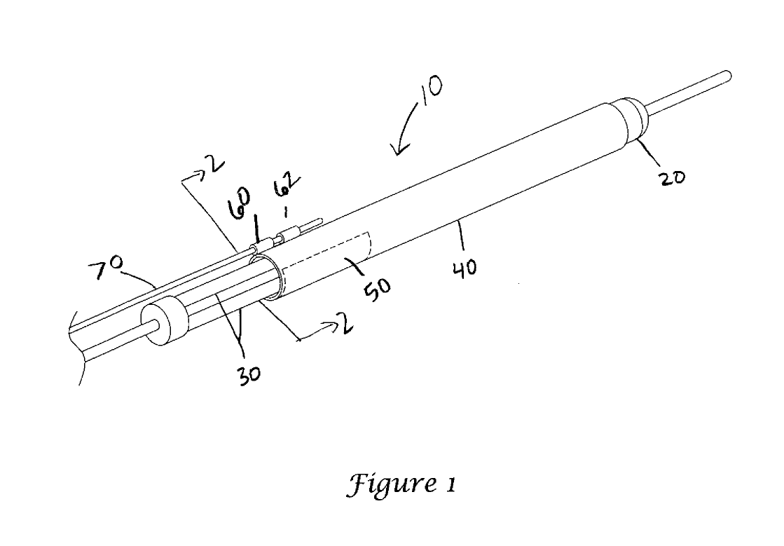

Referring now to FIG. 1 showing a restrained view of a medical device, illustrated is one exemplary embodiment of an intravascular filtering device 10, including filter 20 with a plurality of support struts 30 held in a closed position by sleeve 40. Sleeve 40 acts as a restraining member or mechanism that applies a force against struts 30 to prevent the struts from extending outwardly.

Sleeve 40 surrounds struts 30 and filter 20. Sleeve 40 includes sleeve support band 50 fixedly attached thereto. Sleeve support band 50 may be integrally formed with sleeve 40 or it may be fastened to the sleeve using any available and commonly known means including, but not limited to, adhesives; sonic, vibration, or heat welding; or any of a variety of mechanical fastening techniques. Sleeve support band 50 is biased to expand outwardly from a first position towards a second position. Sleeve support band 50 may be partially constructed of a self-biased spring material or a shape memory material, including but not limited to, shape memory metals such as NiTiNOL; shape memory polymers such as polyurethane; polycycloocetene; cross-linked polyethylene; thermoplastics such as shape memory polyurethanes, polyethylene, polynorborene polymers and copolymers and blends thereof with styrene elastomer copolymers, such as Kraton, and cross-linked transpolyoctylene rubber; cross-linked polyisoprene; styrene butadiene copolymers; bioabsorbable shape memory polymers such as polycaprolactone, copolymers, and/or PLLA PGA copolymers; PMMA; Azodyes, Zwitterionic and other photo chromatic materials. In this exemplary embodiment, sleeve support band 50 may be non-removably coupled to at least one of the plurality of support struts 30.

Sleeve 40 may be formed from a variety of different materials, so long as the material is sufficiently strong to secure struts 30 while being flexible enough to wrap filtering device 10 without damaging any components thereof. For example, sleeve 40 can be fabricated from a metallic film, such as stainless steel or nickel-titanium alloy. Additionally, sleeve 40 may be made from various types of polymer or silicone films, such as but not limited to, heat shrink plastic, nylon, urethane, polymer, low-density polyethylene (LDPE), polyethylene terphthalate (PET), polytetrafluoroethylene (PTFE), fluorinated ethylene propylene (FEP), polyethylene (PE), polyurethane (PU), or silicone tubing.

Further, sleeve 40 may be coated with any one of, or a combination of, substances such as but not limited to, coatings to improve lubricity, reduce platelet aggression, or having anti-thrombogenic properties, hydrophilic coatings, hydrophobic coatings, heparinized coatings, anti-coagulant coatings, Teflon, silicone, medically-useful drugs, or other coatings known to those skilled in the art. Sleeve 40 may also include one or more markers that are visible under fluoroscopy.

With continued reference to FIG. 1, at least two tubular members 60, 62 are fixedly attached to sleeve 40. Tubular members 60, 62 may be integrally formed with sleeve 40 using the same or a different material, or may be fastened to the sleeve using any available and commonly known means including, but not limited to, adhesives; sonic, vibration, or heat welding; or any of a variety of mechanical fastening techniques. Tubular members 60, 62 may be formed from any suitable metal, plastic, or polymer material, and may be coated as needed or desired. Tubular members 60, 62 may include a radiopaque material.

Tubular members 60, 62 are configured to slidably receive actuating member 70 therein. Actuating member 70 may take the form of a wire, rod, or fiber, in any suitable shape or configuration. Actuating member 70 may be formed from any appropriate material, including but not limited to, metals or alloys such as nickel-titanium alloy or stainless steel; any suitable nylon, polyethylene, or any other appropriate polymer or plastic; composites; synthetic materials; or any combination thereof. Actuating member 70 may also include a lubricious or any other appropriate coating.

Material selection of the various components of the restraining mechanism, such as radiopaque materials and/or markers visible under fluoroscopy, can help the physician determine if the device has properly deployed once it has been inserted into the vessel of a patient.

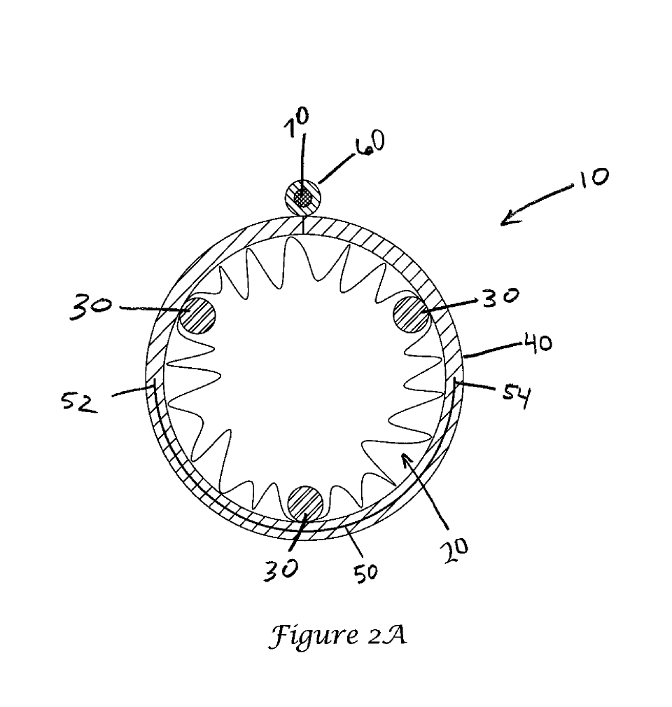

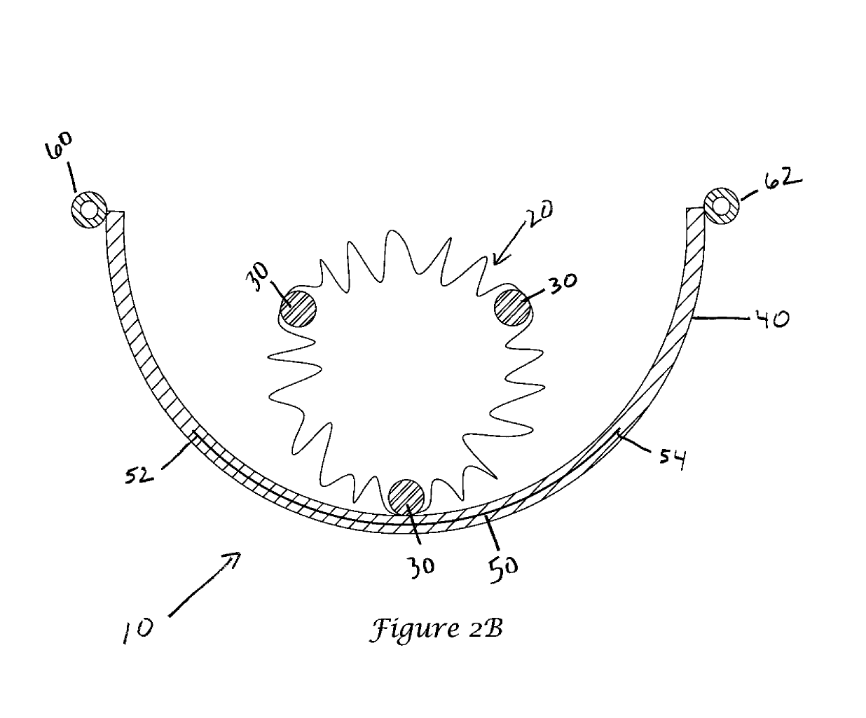

FIG. 2A shows a cross-sectional view of filtering device 10 of FIG. 1. Filtering device 10 can include a plurality of support struts 30 that are biased to extend outwardly. Support struts 30 may be at least partially constructed of a shape memory material, including but not limited to, shape memory metals such as NiTiNOL; shape memory polymers such as polyurethane; polycycloocetene; cross-linked polyethylene; thermoplastics such as shape memory polyurethanes, polyethylene, polynorborene polymers and copolymers and blends thereof with styrene elastomer copolymers, such as Kraton, and cross-linked transpolyoctylene rubber; cross-linked polyisoprene; styrene butadiene copolymers; bioabsorbable shape memory polymers such as polycaprolactone, copolymers, and/or PLLA PGA copolymers; PMMA; Azodyes, Zwitterionic and other photo chromatic materials.

In continuing reference to FIG. 2A, actuating member 70 is slidably engaged with tubular members 60, 62. In this example, sleeve support band 50 has a first end 52 and a second end 54, is integrally formed with sleeve 40, and is shown in a first position. Filter 20 is attached to at least one of support struts 30 and is configured to capture material of a variety of sizes and enable removal of the captured material. Filter 20 has a variety of pores or holes through which fluid can pass, while the size and/or shape of each pore or hole is selected to prevent passage of emboli or other material.

Filter 20 can comprise a microporous membrane formed from a polymeric material. Examples of suitable polymeric materials include polypropylene (PP), polyvinylchloride (PVC), polyamide (nylon), polyurethane, polyester, polyethylene tetraphlalate, polyether-ether ketone (PEEK), polyether block amide (PEBA), polytetraflouroethylene (PTFE), or any mixture, blend or combination thereof. Alternatively, the filter membranes can comprise a woven, braided, or otherwise manufactured mesh screen made from a metallic material such as stainless steel or nickel-titanium alloy.

In reference to FIG. 2B, actuating member 70 has been moved proximal to disengage the actuating member from tubular members 60, 62. This serves to release the restraining force applied to the plurality of support struts 30 by sleeve 40, and allows self-biased sleeve support band 50 to expand outward to a second position, pulling sleeve 40 away from filtering device 10. This will also allow the plurality of support struts 30 to expand outward and deploy filter 20. One benefit of sleeve support band 50 moving sleeve 40 away from filtering device 10 is that filter 20 may be deployed with as little resistance or interference from the sleeve as possible.

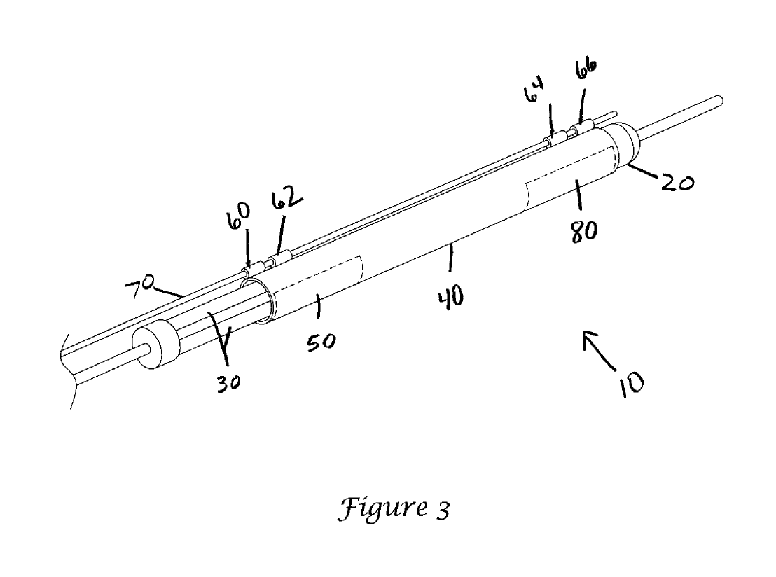

Another exemplary embodiment of the invention is depicted by FIG. 3, and includes structure similar to that of FIG. 1, with the notable addition of a second sleeve support band 80 having a first end and a second end and self-biased to expand outwardly from a first position to a second position, and additional tubular members 64, 66. It should be noted that any suitable number of tubular members 60-66 may be used with any embodiment or configuration of the invention. A second sleeve support band is not required to use tubular members in excess of two, nor are additional tubular members required to use a plurality of sleeve support bands. As in the embodiment of FIG. 1 above, the embodiment of FIG. 3 has tubular members 60-66 engaged by actuating member 70. Tubular members 64, 66 are fixedly attached to sleeve 40 and may be integrally formed with sleeve 40 using the same or a different material, or may be fastened to the sleeve as discussed above with respect to tubular members 60, 62. Tubular members 64, 66 may be formed from any suitable metal, plastic, or polymer material, and may be coated as needed or desired. Tubular members 64, 66 may include a radiopaque material.

In the embodiment depicted by FIG. 3, one sleeve support band is non-removably attached to a support strut. However, it will be appreciated that any configuration may be used—a device with only one of a plurality of sleeve support bands attached to a support strut may be acceptable, a device where any of a plurality of sleeve support bands is attached to different support struts, or a device with none of the sleeve support bands attached to any support strut—throughout any embodiment of the invention.

Tubular members 60-66 are configured to slidably receive actuating member 70 therein. Actuating member 70 may take the form of a wire, rod, or fiber, in any suitable shape or configuration. Actuating member 70 may be formed from any appropriate material as discussed above. Actuating member 70 may also include a lubricious or any other appropriate coating.

Tubular members 60-66 are preferred, but not required, to be oriented in a coaxial fashion. Coaxial orientation improves performance when moving actuating member 70 within tubular members 60-66 by reducing the amount of force required to move the actuating member. However, other orientations of tubular members 60-66 and actuating member 70 may provide acceptable performance, such as but not limited to, having the center bore of the tubular members substantially parallel to one another and having an offset (not shown) in actuating member 70.

Material selection of the various components of the restraining mechanism, such as radiopaque materials and/or markers visible under fluoroscopy, can help the physician determine if the device has properly deployed once it has been inserted into the vessel of a patient.

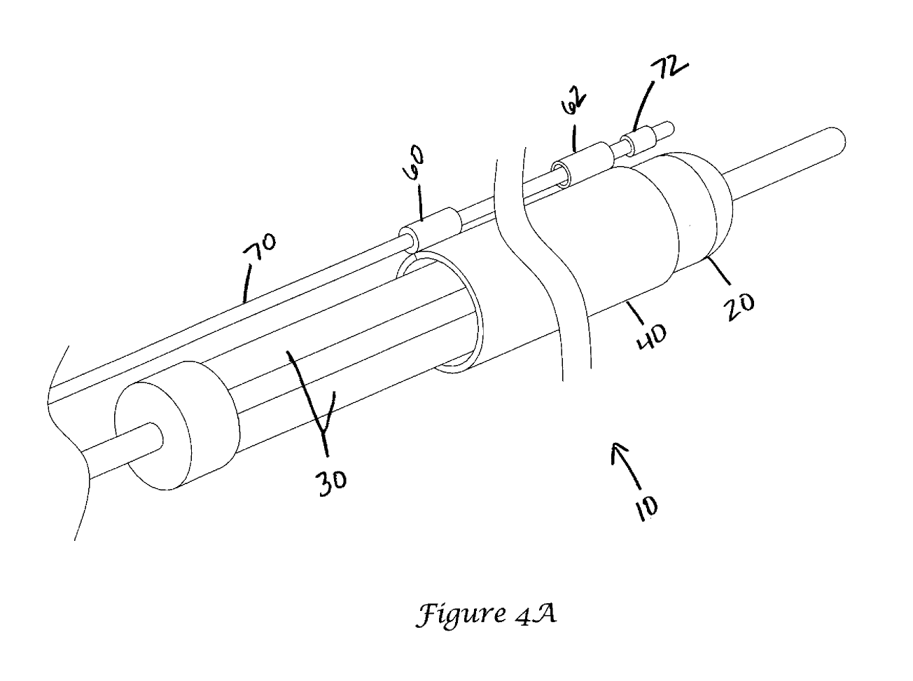

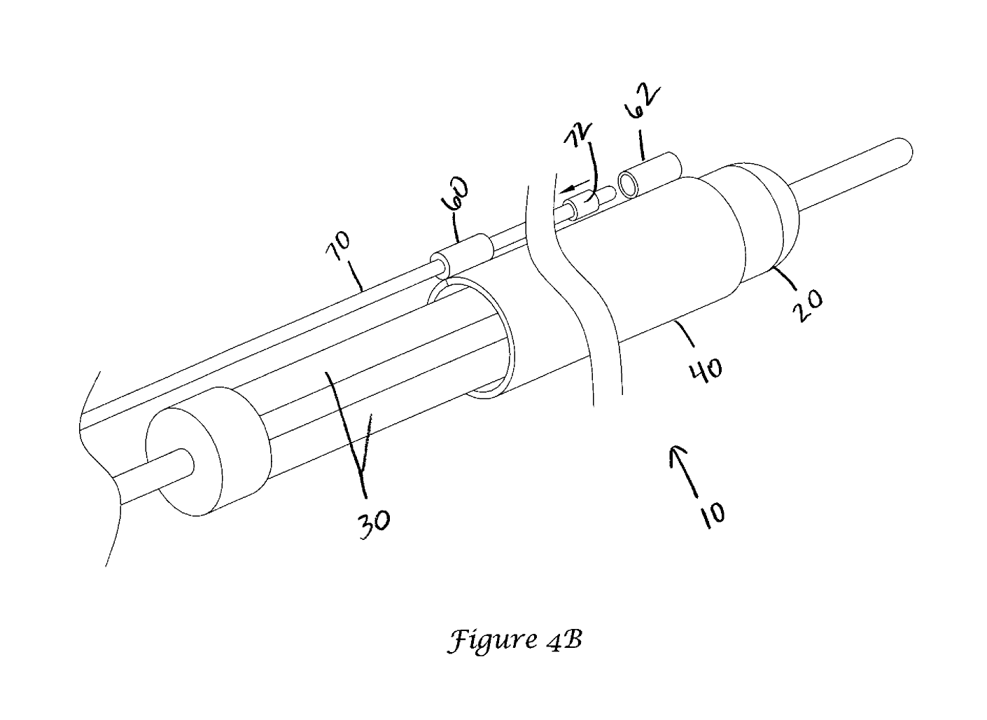

In another exemplary embodiment, a filtering device 10 similar to that of FIG. 1 may have sleeve support band 50 that is not coupled to any of the plurality of support struts 30. As a result, steps must be taken to avoid leaving sleeve 40 free within the vessel of the patient following proximal movement of actuating member 70 and release of the restraining force. Tubular members 60-66 and actuating member 70 are configured so as to retain at least a portion of the sleeve coupled to the actuating member following release of the restraining force. This can be accomplished in a variety of ways, one of which has already been described—using an offset on actuating member such that at least one of the tubular members cannot be disengaged from the actuating member. FIG. 4A illustrates an exemplary means of configuring actuating member 70 and tubular members 60-66.

FIG. 4A shows a detailed view of a portion of an exemplary restraining mechanism including a distal stopping feature 72 as part of actuating member 70. Distal stopping feature 72 and tubular members 60-66 are configured such that the distal stopping feature may pass through one or more tubular members, but may not pass through at least one tubular member. For example, distal stopping feature 72 may have a diameter larger than actuating member 70, but a smaller diameter than the inside of the distalmost tubular members 62 or 62-66 (depending on configuration), and a larger diameter than the inside of the proximalmost tubular member 60. Another example stopping feature (not shown) may include a retractable and/or spring-loaded stopping feature that may pass through tubular member 60 in the distal direction, but cannot be pulled back through in the proximal direction. FIG. 4B shows the exemplary embodiment of FIG. 4A with actuating member 70 moved in a proximal direction to disengage at least one tubular member, while retaining sleeve 40 on the actuating member.

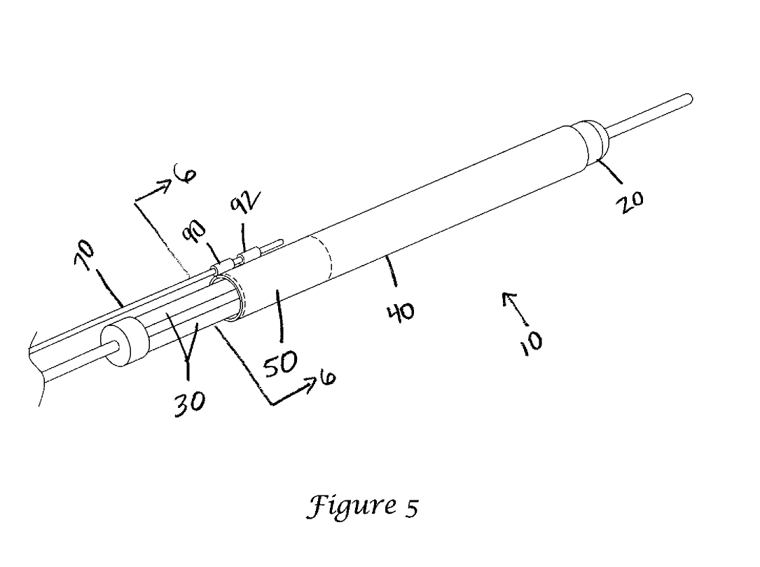

Another exemplary embodiment is illustrated by FIG. 5. FIG. 5 is configured similar to the embodiment of FIG. 1 above, except that sleeve support band 50 has at least two tubular members 90, 92 fixedly attached to the sleeve support band instead of sleeve 40. Tubular members 90, 92 may be integrally formed with sleeve support band 50 using the same or a different material, or may be fastened to the sleeve support band as discussed above with respect to attaching the tubular members 60-66 to sleeve 40. Tubular members 90, 92 may be formed from any suitable metal, plastic, or polymer material, and may be coated as needed or desired. Tubular members 90, 92 may include a radiopaque material.

As in the embodiment of FIG. 1 above, sleeve 40 includes sleeve support band 50 fixedly attached thereto. Sleeve support band 50 may be integrally formed with sleeve 40 or it may be fastened to the sleeve using any available and commonly known means including, but not limited to, adhesives; sonic, vibration, or heat welding; or any of a variety of mechanical fastening techniques. Sleeve support band 50 is biased to expand outwardly from a first position towards a second position. Sleeve support band 50 may be at least partially constructed of a self-biased spring material such as stainless steel and the like, or a shape memory material, including but not limited to, shape memory metals such as NiTiNOL (nickel-titanium alloy); shape memory polymers such as polyurethane; polycycloocetene; cross-linked polyethylene; thermoplastics such as shape memory polyurethanes, polyethylene, polynorborene polymers and copolymers and blends thereof with styrene elastomer copolymers, such as Kraton, and cross-linked transpolyoctylene rubber; cross-linked polyisoprene; styrene butadiene copolymers; bioabsorbable shape memory polymers such as polycaprolactone, copolymers, and/or PLLA PGA copolymers; PMMA; Azodyes, Zwitterionic and other photo chromatic materials. In this exemplary embodiment, sleeve support band 50 may be non-removably coupled to at least one of the plurality of support struts 30.

Sleeve 40 may be formed from a variety of different materials, so long as the material is sufficiently strong to secure struts 30 while being flexible enough to wrap filtering device 10 without damaging any components thereof. For example, sleeve 40 can be fabricated from a metallic film, such as stainless steel or nickel-titanium alloy. Additionally, sleeve 40 may be made from various types of polymer or silicone films, such as but not limited to, heat shrink plastic, nylon, urethane, polymer, low-density polyethylene (LDPE), polyethylene terphthalate (PET), polytetrafluoroethylene (PTFE), fluorinated ethylene propylene (FEP), polyethylene (PE), polyurethane (PU), or silicone tubing.

Further, sleeve 40 may be coated with any one of, or a combination of, substances such as but not limited to, coatings to improve lubricity, reduce platelet aggression, or having anti-thrombogenic properties, hydrophilic coatings, hydrophobic coatings, heparinized coatings, anti-coagulant coatings, Teflon, silicone, medically-useful drugs, or other coatings known to those skilled in the art. Sleeve 40 may also include one or more markers that are visible under fluoroscopy.

Tubular members 90, 92 are configured to slidably receive actuating member 70 therein. Actuating member 70 may take the form of a wire, rod, or fiber, in any suitable shape or configuration. Actuating member 70 may be formed from any appropriate material, including but not limited to, metals or alloys such as nickel-titanium alloy or stainless steel; any suitable nylon, polyethylene, or any other appropriate polymer or plastic; composites; synthetic materials; or any combination thereof. Actuating member 70 may also include a lubricious or any other appropriate coating.

Tubular members 90, 92 are preferred, but not required, to be oriented in a coaxial fashion. Coaxial orientation improves performance when moving actuating member 70 within tubular members 90, 92 by reducing the amount of force required to move the actuating member. However, other orientations of tubular members 90, 92 and actuating member 70 may provide acceptable performance, such as but not limited to, having the center bore of the tubular members substantially parallel to one another and having an offset (not shown) in actuating member 70.

Material selection of the various components of the restraining mechanism, such as radiopaque materials and/or markers visible under fluoroscopy, can help the physician determine if the device has properly deployed once it has been inserted into the vessel of a patient.

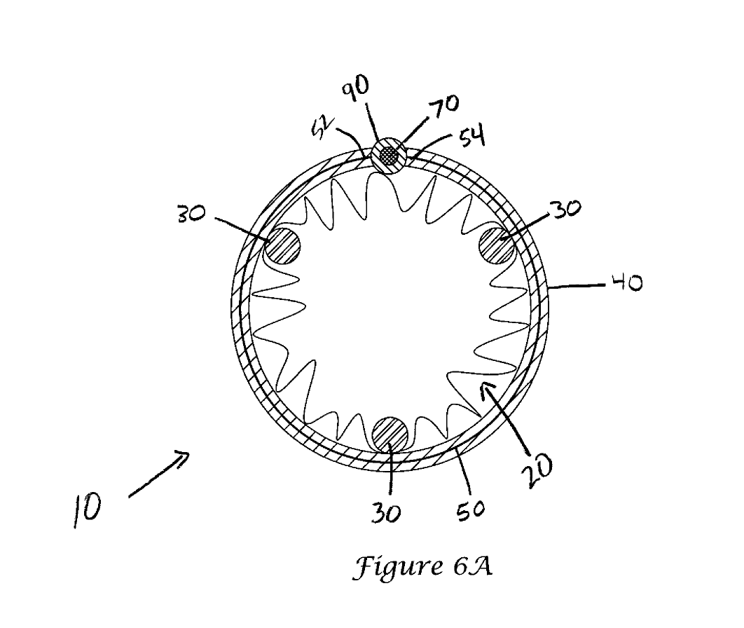

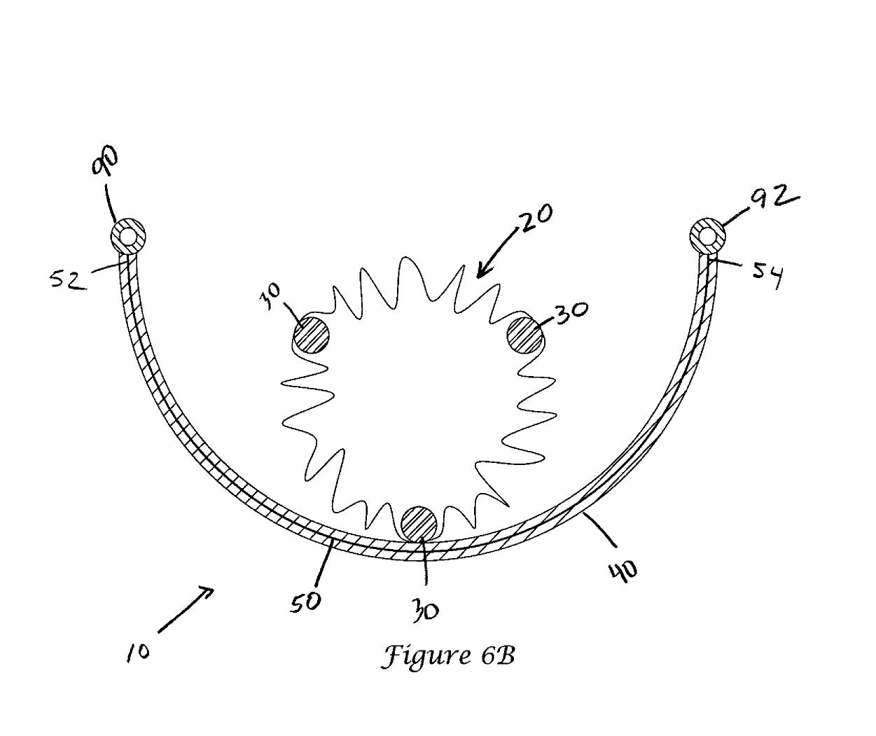

FIG. 6A shows a cross-sectional view of filtering device 10 of FIG. 5. Filtering device 10 can include a plurality of support struts 30 that are biased to extend outwardly. Support struts 30 may be at least partially constructed of a shape memory material, including but not limited to, shape memory metals such as NiTiNOL; shape memory polymers such as polyurethane; polycycloocetene; cross-linked polyethylene; thermoplastics such as shape memory polyurethanes, polyethylene, polynorborene polymers and copolymers and blends thereof with styrene elastomer copolymers, such as Kraton, and cross-linked transpolyoctylene rubber; cross-linked polyisoprene; styrene butadiene copolymers; bioabsorbable shape memory polymers such as polycaprolactone, copolymers, and/or PLLA PGA copolymers; PMMA; Azodyes, Zwitterionic and other photo chromatic materials.

In continuing reference to FIG. 6A, actuating member 70 is slidably engaged with tubular members 90, 92. In this example, sleeve support band 50 has a first end 52 and a second end 54, is integrally formed with sleeve 40, and is shown in a first position. Filter 20 is attached to at least one of support struts 30 and is configured to capture material of a variety of sizes and enable removal of the captured material. Filter 20 has a variety of pores or holes through which fluid can pass, while the size and/or shape of each pore or hole is selected to prevent passage of emboli or other material.

Filter 20 can comprise a microporous membrane formed from a polymeric material. Examples of suitable polymeric materials include polypropylene (PP), polyvinylchloride (PVC), polyamide (nylon), polyurethane, polyester, polyethylene tetraphlalate, polyether-ether ketone (PEEK), polyether block amide (PEBA), polytetraflouroethylene (PTFE), or any mixture, blend or combination thereof. Alternatively, the filter membranes can comprise a woven, braided, or otherwise manufactured mesh screen made from a metallic material such as stainless steel or nickel-titanium alloy.

In reference to FIG. 6B, actuating member 70 has been moved proximal to disengage the actuating member from tubular members 90, 92. This serves to release the restraining force applied to the plurality of support struts 30 by sleeve 40, and allows self-biased sleeve support band 50 to expand outward to a second position, pulling sleeve 40 away from filtering device 10. This will also allow the plurality of support struts 30 to expand outward and deploy filter 20. One benefit of sleeve support band 50 moving sleeve 40 away from filtering device 10 is that filter 20 may be deployed with as little resistance or interference from the sleeve as possible.

Similar to the embodiment described with respect to FIG. 3 above, there may be more than one sleeve support band, including additional tubular members attached to the additional sleeve support band or bands. Tubular members 90, 92 (and any additional tubular members attached to sleeve support bands) may have any, or all, of the characteristics of tubular members 60-66 with regards to composition or materials, attachment means, coatings, or orientation.

Tubular members 90, 92 are configured to slidably receive actuating member 70 therein. Actuating member 70 may take the form of a wire, rod, or fiber, in any suitable shape or configuration. Actuating member 70 may be formed from any appropriate material as discussed above. Actuating member 70 may also include a lubricious or any other appropriate coating.

Tubular members 90, 92 are preferred, but not required, to be oriented in a coaxial fashion. Coaxial orientation improves performance when moving actuating member 70 within tubular members 90, 92 by reducing the amount of force required to move the actuating member. However, other orientations of tubular members 90, 92 and actuating member 70 may provide acceptable performance, such as but not limited to, having the center bore of the tubular members substantially parallel to one another and having an offset (not shown) in actuating member 70.

Material selection of the various components of the restraining mechanism, such as radiopaque materials and/or markers visible under fluoroscopy, can help the physician determine if the device has properly deployed once it has been inserted into the vessel of a patient.

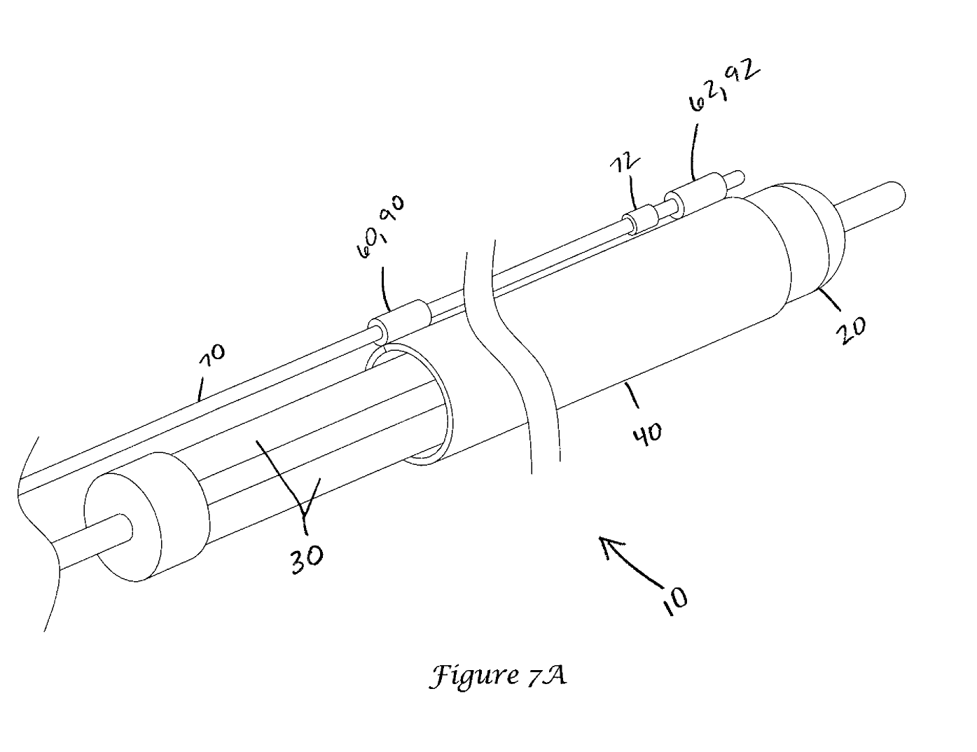

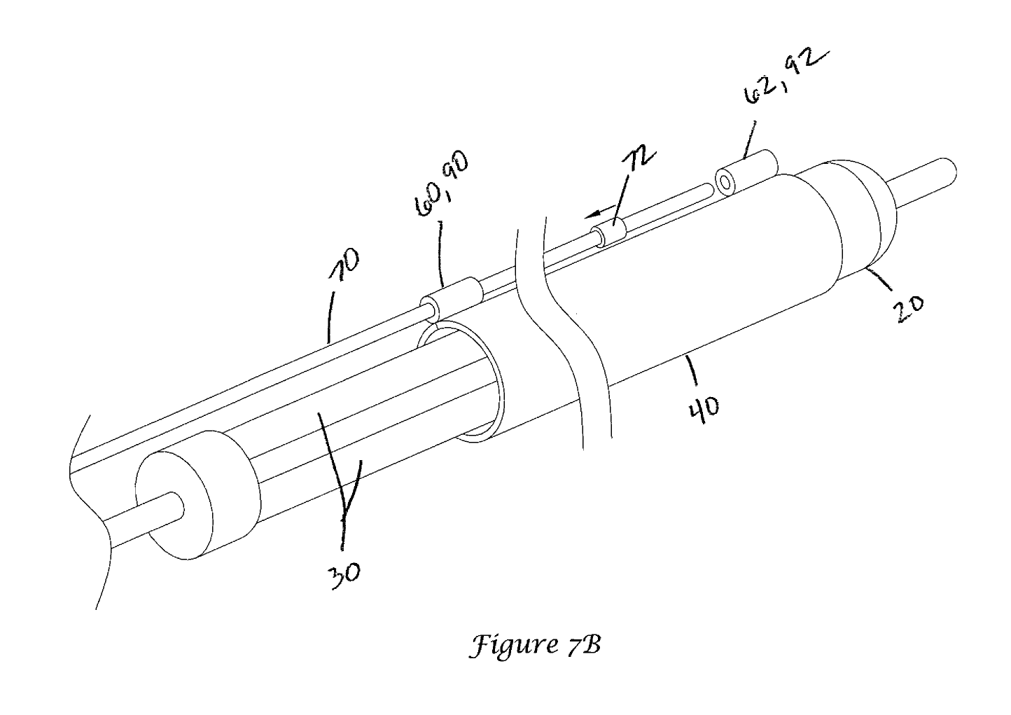

In another exemplary embodiment, a filtering device 10 similar to that of FIG. 5 may have sleeve support band 50 that is not coupled to any of the plurality of support struts 30. As a result, steps must be taken to avoid leaving sleeve 40 free within the vessel of the patient following proximal movement of actuating member 70 and release of the restraining force. Tubular members 60-66 (or 90, 92) and actuating member 70 are configured so as to retain at least a portion of the sleeve coupled to the actuating member following release of the restraining force. This can be accomplished in a variety of ways, one of which has already been described—using an offset on actuating member such that at least one of the tubular members cannot be disengaged from the actuating member. FIG. 7A illustrates an exemplary means of configuring actuating member 70 and tubular members 60-66 (or 90, 92).

FIG. 7A shows a detailed view of a portion of an exemplary restraining mechanism including a stopping feature 72 as part of actuating member 70 located between tubular members 60-66 (or 90, 92 depending on configuration). Placement of stopping feature 72 in this approximate location allows actuating member 70 to disengage tubular members 62-66 (or 92) that are distal the stopping feature while retaining engagement with the tubular member or members 60 (or 90) proximal the stopping feature. An additional benefit of this arrangement is that less movement of actuating member 70 is required to disengage the tubular members. In the event that an offset in actuating member 70 were used, placement of the offset could be similar to that of stopping feature 72. Another example stopping feature (not shown) may include a retractable and/or spring-loaded stopping feature that may pass through tubular member 60 (or 90) in the distal direction, but cannot be pulled back through in the proximal direction. FIG. 7B shows the exemplary embodiment of FIG. 7A with actuating member 70 moved in a proximal direction to disengage at least one tubular member, while retaining sleeve 40 on the actuating member.

It will be appreciated by one skilled in the art that the sleeve retaining means illustrated in FIGS. 4A, 4B, 7A, and 7B may be used in conjunction with any embodiment of the invention, including but not limited to, single or multiple sleeve support bands and any number of tubular members.

1. A restraining mechanism configured to prevent the outward expansion of a plurality of support struts of an intravascular filtering device, the restraining mechanism comprising:
a sleeve having a left side, a right side, and at least two tubular members configured to receive an actuating member;
a sleeve support band having a first end, a second end, a first position, and a second position, the first end and the second end being self-biased to expand from the first position towards the second position; and
an actuating member configured to slidably engage the at least two tubular members;
wherein the sleeve has at least one tubular member fixedly attached to each of the left side and the right side of the sleeve;
wherein the sleeve support band is fixedly attached to the sleeve;
wherein the sleeve is adapted to wrap around the filter and apply a restraining force to the plurality of support struts to prevent the plurality of support struts from expanding outward when the sleeve support band is in the first position; and
wherein the sleeve support band expands towards the second position when the actuating member is moved in a proximal direction.

2. The restraining mechanism of claim 1, wherein the sleeve is formed of a polymer.

3. The restraining mechanism of claim 1, wherein the sleeve is formed of metal.

4. The restraining mechanism of claim 1, wherein the sleeve includes an anti-coagulant, hydrophobic, or hydrophilic coating.

5. The restraining mechanism of claim 1, wherein the sleeve is drug-coated.

6. The restraining mechanism of claim 1, wherein the sleeve includes one or more markers visible under fluoroscopy.

7. The restraining mechanism of claim 1, wherein the sleeve is non-removably coupled to the actuating member.

8. The restraining mechanism of claim 1, wherein the at least two tubular members are formed of a polymer or metal.

9. The restraining mechanism of claim 1, wherein the at least two tubular members are integrally formed with the sleeve.

10. The restraining mechanism of claim 1, wherein the at least two tubular members include a radiopaque material.

11. A restraining mechanism configured to prevent the outward expansion of a plurality of support struts of an intravascular filtering device, the restraining mechanism comprising:
a sleeve;
a sleeve support band having a first end, a second end, a first position, and a second position, the first end and the second end being self-biased to expand from the first position towards the second position;
wherein the sleeve support band is fixedly attached to the sleeve, and having at least one tubular member attached to each of the first end and the second end of the sleeve support band; and
an actuating member configured to slidably engage the tubular members;
wherein the sleeve is adapted to wrap around the filter and apply a restraining force to the plurality of support struts to prevent the plurality of support struts from expanding outward when the sleeve support band is in the first position; and
wherein the actuating member is configured to allow the expansion of the sleeve support band towards the second position when the actuating member is moved in a proximal direction.

12. The restraining mechanism of claim 11, wherein the sleeve is formed of a polymer.

13. The restraining mechanism of claim 11, wherein the sleeve is formed of metal.

14. The restraining mechanism of claim 11, wherein the sleeve includes an anti-coagulant, hydrophobic, or hydrophilic coating.

15. The restraining mechanism of claim 11, wherein the sleeve is drug-coated.

16. The restraining mechanism of claim 11, wherein the sleeve includes one or more markers visible under fluoroscopy.

17. The restraining mechanism of claim 11, wherein the sleeve is non-removably coupled to the actuating member.

18. The restraining mechanism of claim 11, wherein the tubular members are formed of a polymer or metal.

19. The restraining mechanism of claim 11, wherein the tubular members are integrally formed with the sleeve.

20. The restraining mechanism of claim 11, wherein the tubular members include a radiopaque material.
